(19) United States
(12) Patent Application Publication
(10) Pub. No.: US 2025/0164414 A1
(43) Pub. Date: May 22, 2025

(54) RADIOGRAPHY SYSTEM

(71) Applicant: FUJIFILM CORPORATION, Tokyo (JP)

(72) Inventors: Hisatsugu HORIUCHI, Kanagawa (JP); Kyohei MORI, Kanagawa (JP); Ryosuke OGURA, Kanagawa (JP); Masayoshi MATSUURA, Kanagawa (JP); Koji TANINAI, Kanagawa (JP)

(21) Appl. No.: 18/939,554

(22) Filed: Nov. 7, 2024

(30) Foreign Application Priority Data
Nov. 20, 2023 (JP) 2023-196963

(51) Int. Cl. G01N 23/04

(52) U.S. Cl. CPC G01N 23/04; G01N 2223/308; G01N 2223/309

(57) ABSTRACT

A radiography system includes a decubitus table and a radiation source unit that is configured to be independently movable from the decubitus table and includes a radiation source. The radiation source unit is capable of being attached to and detached from the decubitus table, and has an attachment portion that supports at least a part of a weight of the radiation source unit in a state in which the radiation source unit is attached to the decubitus table. The decubitus table has a guide mechanism that guides a movement of the radiation source unit attached through the attachment portion.

### CROSS-REFERENCE TO RELATED APPLICATIONS

The present application claims priority under 35 U.S.C. § 119 to Japanese Patent Application No. 2023-196963, filed on Nov. 20, 2023. The above application is hereby expressly incorporated by reference, in its entirety, into the present application.

### BACKGROUND

### 1. Technical Field

The technology of the present disclosure relates to a radiography system.

### 2. Description of the Related Art

JP2011-136028A discloses a radiography apparatus including a radiation source that outputs radiation, a radiation source main body portion that accommodates the radiation source, a radiation detector that detects the radiation transmitted through a subject and converts the detected radiation into a radiation image in a case in which the radiation source irradiates the subject with the radiation, a cassette main body portion that transmits the radiation and accommodates the radiation detector, and an attachment mechanism that is attached to the radiation source main body portion and is attachable to and detachable from the cassette main body portion or is attachable to and detachable from an imaging table on which the cassette main body portion is disposed.

JP2015-208573A discloses a radiation generation apparatus including a radiation generation unit that generates radiation, a support unit that supports the radiation generation unit, a power supply unit that supplies power to the radiation generation unit, and a moving unit that is provided with the power supply unit and is movable, in which the moving unit is attachable to and detachable from the support unit.

JP2012-50523A discloses a radiation mobile examination cart including a radiation irradiation section that irradiates a subject with radiation from radiation generation unit, a movement section that moves the radiation irradiation section along a predetermined guide, an adjustment section that adjusts an irradiation angle of the radiation to be emitted from the radiation generation unit so that the radiation is radiated to a radiation detection section that moves in a direction opposite to the movement direction of the radiation detection section, in accordance with the movement of the radiation irradiation section by the movement section, and detects the radiation emitted from the radiation irradiation section and transmitted through the subject, and a control section that controls the radiation irradiation section to emit the radiation and controls the radiation detection section to detect the radiation, in synchronization with an irradiation operation of irradiating the radiation from the radiation irradiation section and a detection operation of detecting the radiation by the radiation detection section.

### SUMMARY

JP2011-136028A discloses a technique of fixing a radiation source main body portion that accommodates a radiation source to an imaging table via an attachment mechanism. On the other hand, JP2015-208573A and JP2012-50523A disclose a radiography apparatus including a moving mechanism capable of moving a radiation source with respect to a subject.

However, in the techniques of JP2011-136028A, JP2015-208573A, and JP2012-50523A, the movement of the radiation source along the decubitus table in a state in which the radiation source is attached to the decubitus table is not considered. Therefore, the technology of the present disclosure provides a radiography system capable of easily moving a radiation source unit with respect to a decubitus table while stabilizing the radiation source unit.

A first aspect according to the technology of the present disclosure is a radiography system comprising a decubitus table, and a radiation source unit that is configured to be independently movable from the decubitus table and includes a radiation source, in which the radiation source unit is capable of being attached to and detached from the decubitus table, and has an attachment portion that supports at least a part of a weight of the radiation source unit in a state in which the radiation source unit is attached to the decubitus table, and the decubitus table has a guide mechanism that guides a movement of the radiation source unit attached through the attachment portion.

A second aspect according to the technology of the present disclosure is the radiography system according to the first aspect, in which, in a state in which the radiation source unit is attached to the decubitus table, the radiation source is positioned above the decubitus table, and a position of a centroid of the radiation source unit is positioned on the same side as the radiation source with respect to a side surface of the decubitus table facing the radiation source unit as viewed from a movement direction of the guide mechanism.

A third aspect according to the technology of the present disclosure is the radiography system according to the second aspect, in which the centroid of the radiation source unit is positioned on a radiation source side with respect to the attachment position.

A fourth aspect according to the technology of the present disclosure is the radiography system according to the first aspect, in which, in a state in which the radiation source unit is attached to the decubitus table, the radiation source unit is separated from a floor surface on which the decubitus table is installed.

A fifth aspect according to the technology of the present disclosure is the radiography system according to the first aspect, in which the attachment portion has a first inclined surface that is formed on a distal end side in an attachment direction of the radiation source unit and is inclined upward toward the distal end side.

A sixth aspect according to the technology of the present disclosure is the radiography system according to the first aspect, in which the guide mechanism includes a guide rail that extends along the movement direction of the radiation source unit, and a holding member that is movable along the guide rail, and the attachment portion is attached to the holding member.

A seventh aspect according to the technology of the present disclosure is the radiography system according to the sixth aspect, in which the holding member is a flat plate-shaped stage member, and the attachment portion is attached to the decubitus table in a state in which a lower surface of the attachment portion is placed on an upper surface of the stage member.

An eighth aspect according to the technology of the present disclosure is the radiography system according to the sixth aspect, in which the holding member has a second inclined surface that is formed at an end portion on a radiation source unit side and is inclined downward toward the radiation source unit side.

A ninth aspect according to the technology of the present disclosure is the radiography system according to the sixth aspect, in which the holding member includes a roller member on a surface facing the attachment portion.

A tenth aspect according to the technology of the present disclosure is the radiography system according to the first aspect, in which the radiation source unit has a first site and a second site that is provided on a side of the decubitus table with respect to the first site and is movable in an up-down direction with respect to the first site, and the attachment portion is attachable to and detachable from the decubitus table in a space formed by upward movement of the second site.

An eleventh aspect according to the technology of the present disclosure relates to the radiography system according to the first aspect, in which a placement surface, which is a surface on which the subject is placed in the decubitus table has a rectangular parallelepiped shape in a plan view, and a movement direction of the guide mechanism is along a longitudinal direction of the placement surface.

A twelfth aspect according to the technology of the present disclosure is the radiography system according to the first aspect, in which the guide mechanism is provided on a surface different from a placement surface, which is a surface on which the subject is placed in the decubitus table.

According to the technique of the present disclosure, it is possible to provide a radiography system capable of easily moving a radiation source unit with respect to a decubitus table while stabilizing the radiation source unit.

### BRIEF DESCRIPTION OF THE DRAWINGS

FIG. 1 is a schematic perspective view illustrating an example of a configuration of a radiography system according to an embodiment.

FIG. 2 is a schematic perspective view illustrating an example of the configuration of the radiography system according to the embodiment.

FIG. 3 is a schematic front view illustrating an example of the configuration of the radiography system according to the embodiment.

FIG. 4 is a partially enlarged view illustrating an example of the configuration of the radiography system according to the embodiment.

FIG. 5 is a schematic front view illustrating an example of a configuration of a radiography system according to a comparative example.

FIG. 6 is a schematic front view illustrating an example of the configuration of the radiography system according to the embodiment.

FIG. 7 is a schematic front view illustrating an example of the configuration of the radiography system according to the embodiment.

FIG. 8 is a schematic front view illustrating an example of the configuration of the radiography system according to the embodiment.

FIG. 9 is a partially enlarged view illustrating an example of the configuration of the radiography system according to the embodiment.

### DETAILED DESCRIPTION

An example of an embodiment of a radiography system 10 according to the technique of the present disclosure will be described with reference to the accompanying drawings.

In the following description, for convenience of description, a front-rear direction (also referred to as a depth direction), a width direction, and a height direction of the radiography system 10 are indicated by three arrows X, Y, and Z. First, the height direction is indicated by an arrow Z, an arrow Z direction indicated by the arrow Z is defined as an upper direction of the radiography system 10, and an opposite direction thereof is defined as a lower direction. The width direction is indicated by an arrow X orthogonal to the arrow Z, a direction indicated by the arrow X is defined as a front direction of the radiography system 10, and a direction opposite to the front direction is defined as a rear direction. A direction orthogonal to the arrow Z and the arrow X is indicated by an arrow Y in the right-left direction, a direction indicated by the arrow Y is defined as a left direction of the radiography system 10, and the opposite direction is defined as a right direction. In addition, in the following description, the expression using the side, such as an upper side, a lower side, a left side, a right side, a front side, and a rear side, has the same meaning as the expression using the direction.

In the present embodiment, a “vertical direction” refers not only to a perfect vertical direction but also to a vertical direction in the sense of including an error that is generally acceptable in the technical field to which the technology of the present disclosure belongs and that does not contradict the concept of the technology of the present disclosure. In addition, similarly, “horizontal direction” refers to the horizontal direction in the sense of including an error generally allowed in the technical field to which the technology of the present disclosure belongs, that is, an error to the extent that it does not contradict the gist of the technology of the present disclosure, in addition to the exact horizontal direction.

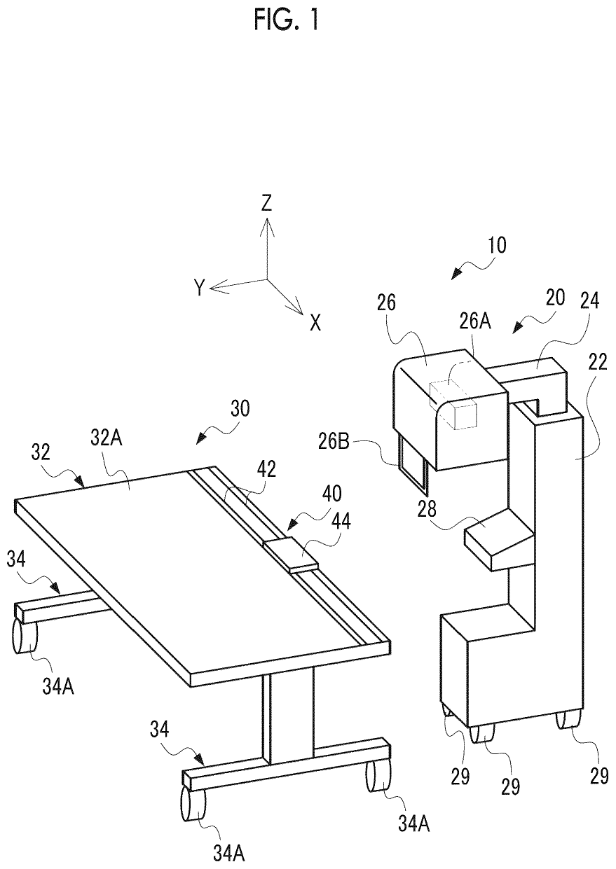

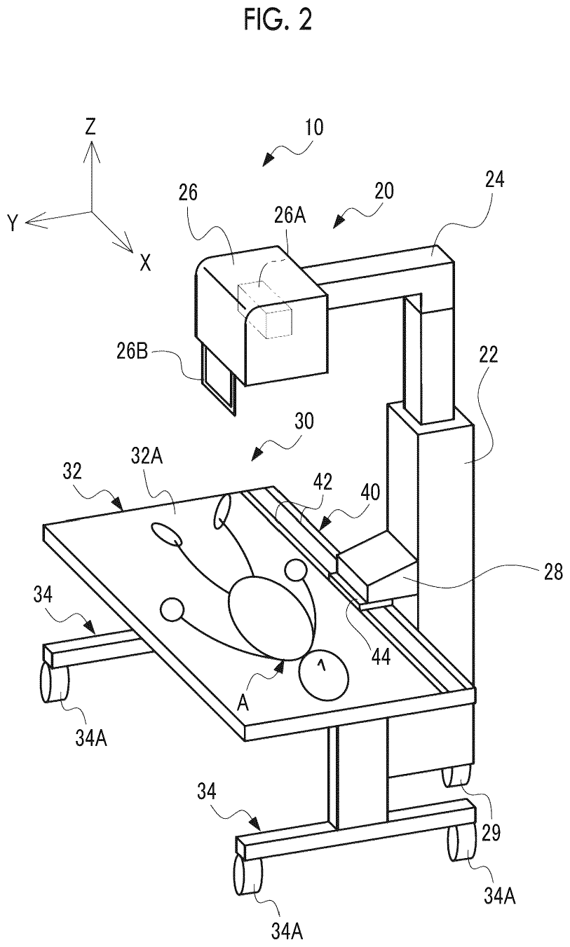

As shown in FIG. 1 as an example, a radiography system 10 is a system that performs radiography on a subject A (see FIG. 2). The radiography system 10 includes a radiation source unit 20 and a decubitus table 30. The radiation source unit 20 is a device that comprises a radiation source 26A and irradiates the subject A with radiation (for example, X-rays or gamma rays) generated from the radiation source 26A. The decubitus table 30 is a table on which the subject A can be placed. In a state in which the subject A is placed on the decubitus table 30, the subject A is irradiated with radiation, and radiography is performed. The radiography system 10 is an example of a “radiography system” according to the technique of the present disclosure. The radiation source unit 20 is an example of a “radiation source unit” according to the technique of the present disclosure. The decubitus table 30 is an example of a “decubitus table” according to the technique of the present disclosure.

The radiation source unit 20 comprises a main body portion 22, an arm 24, and a radiation irradiation portion 26. The main body portion 22 is a portion forming a body portion of the radiation source unit 20, and accommodates a power source system that supplies power to the radiation source 26A inside, a control device that controls the entire radiation source unit 20, a mechanism for driving the arm 24, and the like.

The arm 24 is a part extending from the main body portion 22, and one end thereof is attached to the main body portion 22, and the radiation irradiation portion 26 is provided at the other end thereof. The arm 24 allows the radiation irradiation portion 26 to be movable with respect to the subject A. The arm 24 can be extended and contracted in the up-down direction and can also be extended and contracted in the right-left direction. With this configuration, the radiation irradiation portion 26 can be moved with respect to the subject A through the arm 24.

In the example shown in FIG. 1, the arm 24 extends from an upper surface of the main body portion 22. Specifically, the arm 24 has an L-shape having a part extending along an up-down direction from an upper surface of the main body portion 22 and a part bent from an upper end of the part and extending along a right-left direction. Here, although a form example has been described in which the arm 24 has an L-shape, this is merely an example. The arm 24 may be a C-shaped arm as viewed from the front-rear direction or may be an arm having a plurality of joints.

The radiation irradiation portion 26 irradiates the subject A with radiation. The radiation irradiation portion 26 is attached to a distal end of the arm 24. The radiation source 26A is accommodated in the radiation irradiation portion 26. An irradiation direction of the radiation generated in the radiation source 26A is defined by an irradiation field limiter (not shown) inside the radiation source 26A. Then, the radiation source 26A emits radiation to the subject A. In the example illustrated in FIG. 1, the radiation irradiation portion 26 emits radiation downward. The radiation source 26A is an example of a “radiation source” according to the technology of the present disclosure.

In addition, the radiation irradiation portion 26 comprises a gripping portion 26B. The gripping portion 26B is a portion that can be gripped by a user (for example, a radiologist or a doctor) of the radiography system 10. Although the details will be described later, the user can move the radiation irradiation portion 26 with respect to the decubitus table 30 by gripping the gripping portion 26B.

The attachment portion 28 is provided in an intermediate portion of the main body portion 22 in an up-down direction. The attachment portion 28 is a portion to which the radiation source unit 20 can be attached to and detached from the decubitus table 30. That is, the radiation source unit 20 is attached to the decubitus table 30 through the attachment portion 28. In the example shown in FIG. 1, the attachment portion 28 is a portion that protrudes from a left side surface of the main body portion 22. The position where the attachment portion 28 is provided is merely an example, and the attachment portion 28 may be provided on the front side surface or the rear side surface of the main body portion 22. Details of the attachment portion 28 will be described later. The attachment portion 28 is an example of an “attachment portion” according to the technique of the present disclosure.

In addition, wheels 29 are provided on the lower surface of the main body portion 22. The radiation source unit 20 can travel independently with respect to the decubitus table 30 through the wheels 29 provided in the main body portion 22. The radiation source unit 20, for example, may travel through the wheels 29 by being pushed by the user, or may travel by receiving power from a power source (for example, a motor) (not shown) to rotate the wheels 29. In the example shown in FIG. 1, the wheels 29 are provided at four corners of the lower surface of the main body portion 22. The wheel 29 is, for example, a caster.

The decubitus table 30 comprises a placement portion 32, a leg portion 34, and a guide mechanism 40. The placement portion 32 is a flat plate-shaped portion on which the subject A is placed. In the example shown in FIG. 1, the placement portion 32 has a rectangular parallelepiped shape having a front-rear direction as a longitudinal direction. The placement portion 32 has a placement surface 32A on which the subject A is placed, on an upper side. That is, the placement surface 32A has a rectangular parallelepiped shape in a plan view. The decubitus table 30 is, for example, a dedicated bed for radiography. The decubitus table 30 is provided with a radiation detector (not shown), detects radiation emitted from the radiation source 26A and transmitted through a part of the subject A to be diagnosed, and outputs a radiation image. The radiation detector is referred to as a flat panel detector (FPD).

In addition, leg portions 34 are provided on the lower surface of the placement portion 32. The leg portions 34 are members that support the placement portion 32. The placement portion 32 is set to a predetermined height from the floor surface by the leg portions 34. In the example shown in FIG. 1, the leg portions 34 are provided at both ends of the placement portion 32 in the front-rear direction, respectively. Wheels 34A are provided in the pair of leg portions 34, respectively. In the example shown in FIG. 1, one wheel 34A is provided at each of both end portions of the leg portion 34 in the right-left direction. The decubitus table 30 can be moved by the wheels 34A. The decubitus table 30 may be configured to have no wheel 34A and be fixed to a floor surface.

The guide mechanism 40 is a mechanism that allows the radiation source unit 20 attached through the attachment portion 28 to be movable with respect to the decubitus table 30. Specifically, the guide mechanism 40 is a mechanism for guiding the movement along one side of the placement surface 32A of the radiation source unit 20. The guide mechanism 40 is an example of a “guide mechanism” according to the technique of the present disclosure.

More specifically, the guide mechanism 40 is provided in the placement portion 32. The guide mechanism 40 comprises a pair of guide rails 42 and a stage member 44 guided by the pair of guide rails 42. In the example shown in FIG. 1, the guide mechanism 40 is provided at a right end portion of the placement surface 32A of the placement portion 32. The pair of guide rails 42 extend along the longitudinal direction (here, the front-rear direction) of the placement surface 32A, and the stage member 44 is freely movable along the longitudinal direction of the placement surface 32A along the guide rails 42. That is, the movement direction of the radiation source unit 20 by the guide mechanism 40 is along the longitudinal direction of the placement surface 32A. The pair of guide rails 42 are an example of a “guide rail” according to the technique of the present disclosure, and the stage member 44 is an example of a “holding member” and a “stage member” according to the technique of the present disclosure.

As shown in FIG. 2 as an example, radiography is performed on a subject A placed on the decubitus table 30. In this case, the arm 24 is extended in order to dispose the radiation source 26A at a predetermined position with respect to the subject A. Specifically, the arm 24 is extended upward with respect to the main body portion 22 or is extended toward the side of the decubitus table 30 (here, toward the left side). As a result, the radiation source 26A is disposed at a predetermined position with respect to the subject A.

Here, in a case in which the arm 24 is extended to move the radiation source 26A, the radiation irradiation portion 26 is also moved accordingly. The radiation irradiation portion 26 is provided with various mechanisms and electronic devices in addition to the radiation source 26A. Therefore, the centroid of the radiation source unit 20 also moves with the movement of the radiation irradiation portion 26. For example, in a case in which the arm 24 is extended for radiography and the radiation source 26A is positioned above the decubitus table 30, the centroid of the radiation source unit 20 is moved above the decubitus table 30 and to the decubitus table 30 side (here, the left side). Therefore, it is considered that the centroid of the radiation source unit 20 is separated from the main body portion 22, and the posture of the radiation source unit 20 is unstable (for example, the radiation source unit 20 is likely to fall or waver during movement). In particular, since the radiation source unit 20 according to the present embodiment can travel through the wheels 29, the influence of the instability is larger than that of a stationary radiation irradiation apparatus fixed to a floor surface, a ceiling, or the like.

Therefore, in the radiography system 10 according to the present embodiment, in a case in which radiography is performed, the radiation source unit 20 is attached to the decubitus table 30 through the attachment portion 28. In a case in which the radiography is performed, the radiation source 26A is positioned above the decubitus table 30 in a state in which the radiation source unit 20 is attached to the decubitus table 30. Since the decubitus table 30 has a sufficient weight with respect to the radiation source unit 20 and the position of the centroid is low, the posture of the radiation source unit 20 is stabilized even in a case where the arm 24 is extended by attaching the radiation source unit 20 to the decubitus table 30.

In a case in which the radiography is performed, the radiation source 26A may be moved in the front-rear direction (the direction along the X direction shown in FIG. 2) in addition to the right-left direction and the up-down direction. In a case where the radiation source 26A is moved in the front-rear direction, the user moves the radiation source 26A in the front-rear direction by gripping the gripping portion 26B provided in the radiation irradiation portion 26. In this case, the entire radiation source unit 20 is also moved in the front-rear direction in accordance with the operation by the user, but the radiation source unit 20 has a relatively large weight, and thus it may be difficult for the user to grip the gripping portion 26B and move the radiation source unit 20.

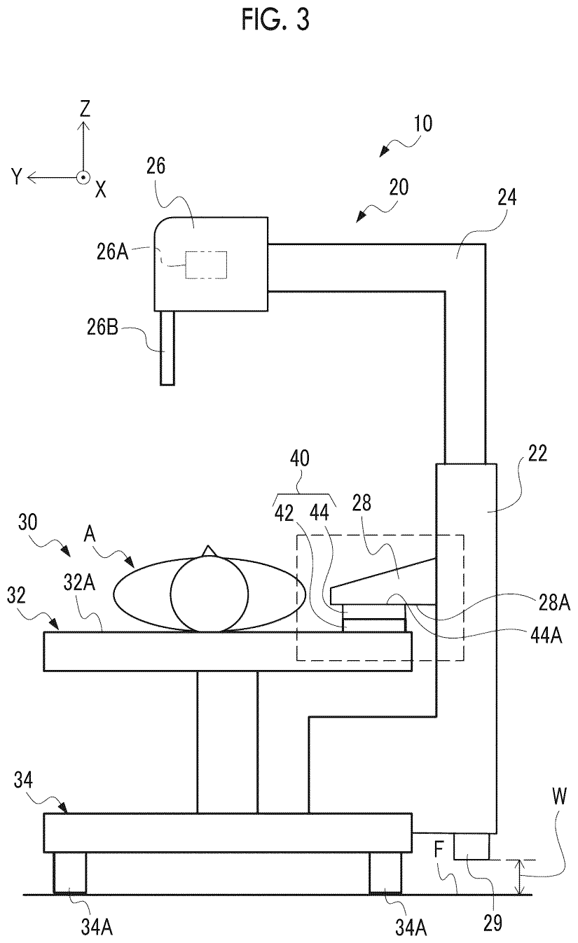

Therefore, as shown in FIG. 3 as an example, the attachment portion 28 supports at least a part of the weight of the radiation source unit 20 in a state in which the radiation source unit 20 is attached to the decubitus table 30. The attachment portion 28 is attached to a guide mechanism 40 of the decubitus table 30. Specifically, the attachment portion 28 is attached to a stage member 44 of the guide mechanism 40. In the example shown in FIG. 3, the attachment portion 28 is a portion that protrudes from the main body portion 22 and is a portion having a trapezoidal shape as viewed from the front-rear direction. In addition, the stage member 44 is a flat plate-shaped member. Then, the attachment portion 28 is attached to the stage member 44 in a state where the lower surface 28A of the attachment portion 28 is placed on the upper surface 44A of the stage member 44.

The load applied by the radiation source unit 20 is transmitted to the stage member 44 through the attachment portion 28. The load applied by the radiation source unit 20 is transmitted to the decubitus table 30. In this way, the weight of the radiation source unit 20 is supported by the attachment portion 28. In the example shown in FIG. 3, the entire weight of the radiation source unit 20 is supported by the attachment portion 28. In this case, the radiation source unit 20 is separated from the floor surface F. That is, a gap W is formed between the lower surface of the wheel 29 of the radiation source unit 20 and the floor surface F. The floor surface F is an example of a “floor surface” according to the technique of the present disclosure.

Here, although a form example has been described in which the entire weight of the radiation source unit 20 is supported by the attachment portion 28, the technology of the present disclosure is not limited to this. An aspect in which approximately 80% of the weight of the radiation source unit 20 is supported by the attachment portion 28 and the remaining approximately 20% is supported by the wheel 29 may be adopted.

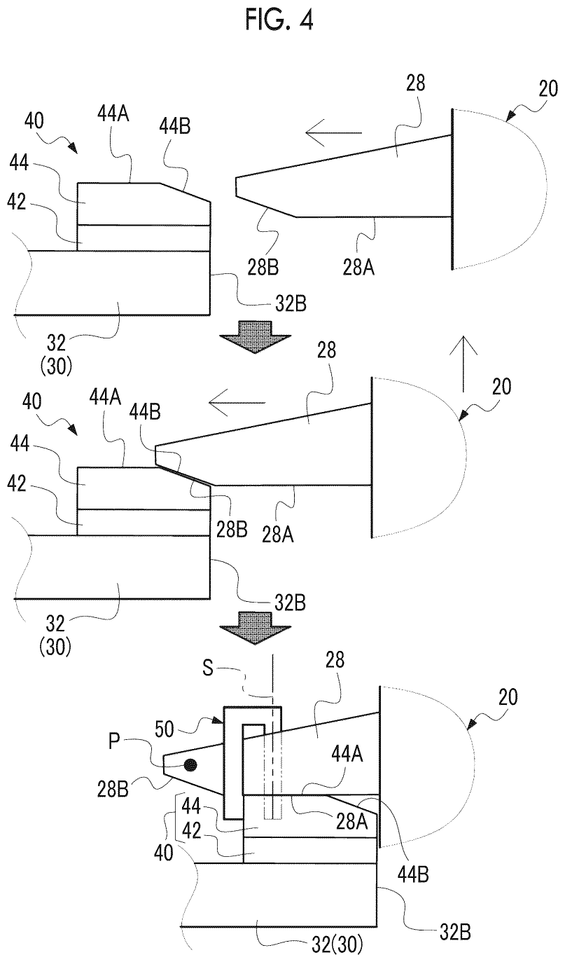

Next, a description will be made of a state where the attachment portion 28 is attached to the stage member 44 with reference to FIG. 4. FIG. 4 is a partially enlarged view of the dotted line frame in FIG. 3. As shown in the upper part of FIG. 4 as an example, first, in a state before the attachment of the radiation source unit 20, the radiation source unit 20 is moved toward the decubitus table 30. Here, the attachment portion 28 is positioned to face the stage member 44 in the movement direction of the radiation source unit 20. Therefore, in a case where the radiation source unit 20 is moved toward the decubitus table 30, the attachment portion 28 comes into contact with the stage member 44. Here, the movement direction in a case where the radiation source unit 20 is attached (hereinafter, simply referred to as an “attachment direction”) is the left direction.

Next, as shown in the middle part of FIG. 4, in a case where the attachment portion 28 comes into contact with the stage member 44, the attachment portion 28 continues to move while being in contact with the stage member 44 as it is. Here, an inclined surface 28B is formed on the attachment portion 28 on the distal end side in an attachment direction. The inclined surface 28B is a surface that is inclined upward toward the distal end side of the attachment portion 28. In the example shown in FIG. 4, the inclined surface 28B is formed on a left end portion of the lower surface 28A of the attachment portion 28. The inclined surface 28B is a surface that is inclined upward toward the left end. The inclined surface 28B is an example of a “first inclined surface” according to the technique of the present disclosure.

In addition, the stage member 44 is formed with an inclined surface 44B at an end portion on the radiation source unit 20 side in the attachment direction. The inclined surface 44B is a surface that is inclined downward toward the radiation source unit 20 side. In the example shown in FIG. 4, the inclined surface 44B is formed on a right end portion of an upper surface 44A of the stage member 44. The inclined surface 44B is a surface that is more inclined downward toward the right side. The inclined surface 44B is an example of a “second inclined surface” according to the technique of the present disclosure.

In a case where the attachment portion 28 comes into contact with the stage member 44, first, the inclined surface 28B of the attachment portion 28 comes into contact with the inclined surface 44B of the stage member 44. Then, the attachment portion 28 is moved with respect to the stage member 44 while the inclined surface 28B and the inclined surface 44B slide. In this case, since the attachment portion 28 moves in the upper left direction along the inclined surface 44B, the entire radiation source unit 20 also moves in the upper left direction together with the attachment portion 28. That is, the radiation source unit 20 moves not only in the attachment direction but also in the upward direction. As a result, the radiation source unit 20 is in a state of being separated from the floor surface F (see FIG. 3).

As shown in the lower part of FIG. 4, in a case where the movement of the attachment portion 28 in the attachment direction is continued, the sliding between the inclined surface 28B of the attachment portion 28 and the inclined surface 44B of the stage member 44 is completed. Then, the attachment portion 28 overcomes the inclined surface 44B, and the movement in the attachment direction is continued as it is. In a case where the movement of the attachment portion 28 in the attachment direction is completed, the lower surface 28A of the attachment portion 28 is placed on the upper surface 44A of the stage member 44. In this state, the attachment portion 28 is fixed to the stage member 44 by the locking member 50. For example, the locking member 50 is a U-shaped member, and one of the pair of rod-shaped members penetrates the attachment portion 28 and the stage member 44. Accordingly, the attachment portion 28 and the stage member 44 are fixed to each other. As described above, the radiation source unit 20 is attached to the decubitus table 30 through the attachment portion 28. The radiation source unit 20 is movable by the guide mechanism 40 in a state of being attached to the decubitus table 30 through the attachment portion 28.

The above-described locking mechanism is merely one example. It is sufficient that the attachment portion 28 and the stage member 44 can be fixed to each other, and the attachment portion 28 and the stage member 44 may be fixed to each other in a manner of being sandwiched by a clamp member in the up-down direction or may be fixed by utilizing magnetic attraction between the lower surface 28A and the upper surface 44A.

In addition, in a state in which the radiation source unit 20 is attached to the decubitus table 30, the position of the centroid P of the radiation source unit 20 is positioned on the radiation source 26A side with respect to a right side surface 32B of the placement portion 32 of the decubitus table 30 as viewed from the movement direction of the guide mechanism 40 (here, the front-rear direction). Here, the position of the centroid P is positioned on the left side of the right side surface 32B. That is, the position of the centroid P of the radiation source unit 20 is positioned on the radiation source 26A side with respect to the surface of the decubitus table 30 on the radiation source unit 20 side.

Further, in a state in which the radiation source unit 20 is attached to the decubitus table 30, a position of the centroid P of the radiation source unit 20 is positioned on a radiation source 26A side with respect to the attachment position S as viewed from the movement direction of the radiation source unit 20 by the guide mechanism 40. Here, the attachment position S is a position at which the attachment portion 28 and the stage member 44 are fixed in a case where the movement direction of the radiation source unit 20 by the guide mechanism 40 is viewed. In a case where the attachment portion 28 and the stage member 44 are fixed to each other by surface contact, the attachment position S is an intermediate position of the contact region in a case where the movement direction of the radiation source unit 20 by the guide mechanism 40 is viewed.

In a state in which the radiation source unit 20 is attached to the decubitus table 30 in this way, the user moves the radiation source unit 20. Specifically, the user stands on the left side of the decubitus table 30 and grips the gripping portion 26B of the radiation irradiation portion 26. Then, the radiation source unit 20 is moved in the front-rear direction through the gripping portion 26B. In this case, the weight of the radiation source unit 20 is supported by the attachment portion 28. In addition, the attachment portion 28 is attached to the stage member 44 of the guide mechanism 40, and the radiation source unit 20 is moved along the pair of guide rails 42 in the front-rear direction. As a result, the user adjusts the position of the radiation source 26A in the front-rear direction.

As described above, the radiography system 10 according to the present embodiment includes the radiation source unit 20 and the decubitus table 30. The radiation source 26A is provided in the radiation source unit 20, and the radiation source unit 20 can travel independently with respect to the decubitus table 30. The radiation source unit 20 that can travel independently with respect to the decubitus table 30 is attached to the decubitus table 30, so that the posture of the radiation source unit 20 is stabilized. For example, even in a case where the arm 24 is extended along the up-down direction, the posture of the radiation source unit 20 is stabilized. Here, the radiation source unit 20 attached to the decubitus table 30 needs to be moved along the decubitus table 30 for position adjustment, but it is difficult to move the radiation source unit 20 because the radiation source unit 20 has a weight.

In the present configuration, at least a part of the weight of the radiation source unit 20 is supported by the attachment portion 28 of the radiation source unit 20. Further, the guide mechanism 40 provided on the decubitus table 30 allows the radiation source unit 20 to be movable along the decubitus table 30. Accordingly, it is easy to move the radiation source unit 20 with respect to the decubitus table 30 while stabilizing the posture of the radiation source unit 20.

In addition, in the radiography, in a case in which the position of the radiation source 26A in the direction along the decubitus table 30 (here, the front-rear direction) is adjusted, a fine position adjustment may be required. In this case as well, since the weight of the radiation source unit 20 is present, it is difficult to move the radiation source unit 20 and perform fine position adjustment. In the present configuration, since at least a part of the weight of the radiation source unit 20 is supported by the attachment portion 28, it is easy to move the radiation source unit 20. Therefore, it is possible to perform fine position adjustment of the radiation source unit 20.

In addition, in the radiography system 10 according to the present embodiment, in a state in which the radiation source unit 20 is attached to the decubitus table 30, the radiation source 26A is positioned above the decubitus table 30, and a position of the centroid P of the radiation source unit 20 is positioned on the same side as the radiation source 26A with respect to a side surface 32B of the decubitus table 30 facing the radiation source unit 20 in a case where the movement direction by the guide mechanism 40 (for example, a front-rear direction) is viewed. Here, the user often moves the radiation source unit 20 by gripping the gripping portion 26B of the radiation irradiation portion 26 from the side opposite to the side to which the radiation source unit 20 is attached (here, the left side). In the present configuration, since the centroid P of the radiation source unit 20 is on the same side as the radiation source 26A with respect to the side surface 32B of the decubitus table 30 to which the radiation source unit 20 is attached, the position of the centroid P is close to the action point. As a result, the user can easily perform the movement operation on the radiation source unit 20 in a state of being attached to the decubitus table 30. In addition, in the radiography system 10, space saving is realized on a side opposite to the decubitus table 30 with respect to the radiation source unit 20 (here, a right side).

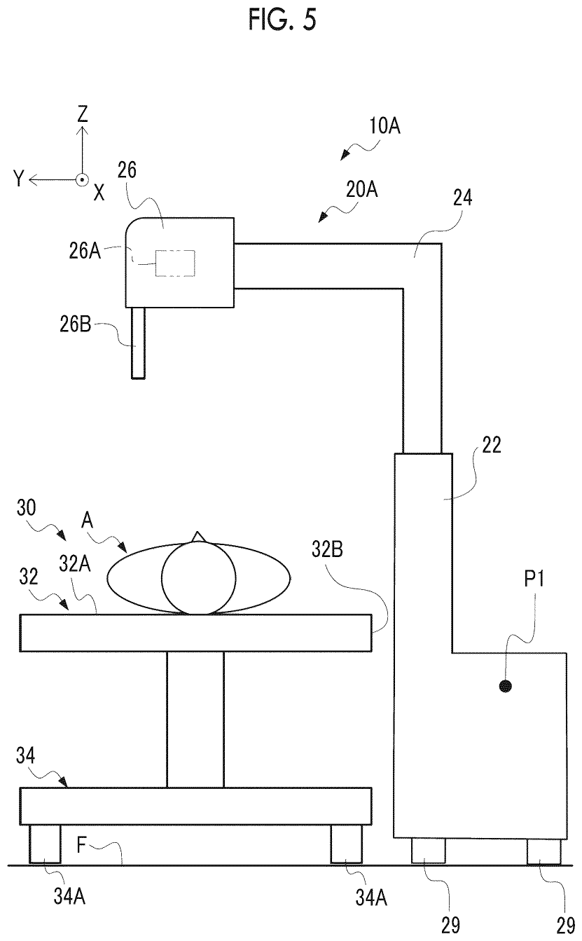

For example, as a comparative example, the radiation source unit 20A in the radiography system 10A will be considered as shown in FIG. 5. In the radiation source unit 20A, the lower part of the main body portion 22 protrudes to the side opposite to the decubitus table 30 (here, the right side). In this case, the centroid P1 of the radiation source unit 20A is positioned on the right side of the portion along the up-down direction of the arm 24. That is, the centroid P1 of the radiation source unit 20A is positioned on the side opposite to the radiation source 26A with respect to the side surface 32B of the decubitus table 30. In this case, in a case in which the radiation source unit 20A is moved by gripping the gripping portion 26B, it is difficult to move the radiation source unit 20A because the position of the action point is separated from the position of the centroid P1. On the other hand, in the present configuration, since the position of the centroid P is positioned on the same side as the radiation source 26A with respect to the side surface 32B of the decubitus table 30, the position of the centroid P and the action point are closer than in the comparative example. Therefore, it is easy to move the radiation source unit 20A in a state of being attached to the decubitus table 30.

In addition, in the radiography system 10 according to the present embodiment, the position of the centroid P of the radiation source unit 20 is positioned on the radiation source 26A side with respect to the attachment position S in a case where the movement direction by the guide mechanism 40 (for example, the front-rear direction) is viewed. Accordingly, since the centroid P is closer to the radiation source 26A, the movement operation of the radiation source unit 20A in a state of being attached to the decubitus table 30 is facilitated.

In addition, in the radiography system 10 according to the present embodiment, in a state in which the radiation source unit 20 is attached to the decubitus table 30, the radiation source unit 20 is separated from the floor surface F on which the decubitus table 30 is installed. Accordingly, the resistance between the radiation source unit 20 and the floor surface F is eliminated, and the radiation source unit 20 is moved by the guide mechanism 40. Therefore, the radiation source unit 20 is easily moved.

In addition, in the radiography system 10 according to the present embodiment, the attachment portion 28 is formed with an inclined surface 28B that is formed on a surface formed on the distal end side in the attachment direction of the radiation source unit 20 and is inclined upward toward the distal end side. Accordingly, in a case where the radiation source unit 20 is attached to the decubitus table 30, the radiation source unit 20 rises with respect to the decubitus table 30 along the inclined surface 28B as it is. As a result, the resistance between the floor surface F and the radiation source unit 20 is reduced, and the radiation source unit 20 can be easily moved after being attached to the decubitus table 30.

In addition, in the radiography system 10 according to the present embodiment, the guide mechanism 40 is provided with the decubitus table 30. The guide mechanism 40 includes a guide rail 42 that extends along the movement direction of the radiation source unit 20, and a stage member 44 that is movable along the guide rail 42. The attachment portion 28 is attached to the stage member 44. Accordingly, the stage member 44 to which the attachment portion 28 is attached can be moved along the guide rail 42. As a result, the movement of the radiation source unit 20 is stabilized, and the movement direction is defined.

In addition, in the radiography system 10 according to the present embodiment, the stage member 44 has a flat plate shape, and the attachment portion 28 is attached to the decubitus table 30 in a state in which the lower surface 28A of the attachment portion 28 is placed on the upper surface 44A of the stage member 44. Accordingly, since the attachment portion 28 is supported from below by the stage member 44, it is easy to support the weight of the radiation source unit 20.

In addition, in the radiography system 10 according to the present embodiment, the stage member 44 is formed with an inclined surface 44B that is a surface formed at an end portion on the side of the radiation source unit 20 and is inclined downward toward the radiation source unit 20 side. Accordingly, in a case in which the radiation source unit 20 is attached to the decubitus table 30, the radiation source unit 20 rises with respect to the decubitus table 30 along the inclined surface 44B as it is. As a result, the resistance between the floor surface F and the radiation source unit 20 is reduced, and the radiation source unit 20 can be easily moved after being attached to the decubitus table 30.

In addition, in the radiography system 10 according to the present embodiment, the placement surface 32A, which is a surface on which the subject A is placed in the decubitus table 30, has a rectangular parallelepiped shape in a plan view (that is, in a case of being viewed from above), and the movement direction of the radiation source unit 20 by the guide mechanism 40 is along the longitudinal direction of the placement surface 32A. Accordingly, the amount of movement by the guide mechanism 40 is longer than the amount of movement in a case of the movement along the lateral direction. Therefore, the effect of facilitating the movement of the radiation source unit 20 is further exerted.

### First Modification Example

In the embodiment described above, the form example has been described in which the guide mechanism 40 is provided on the placement surface 32A, but the technology of the present disclosure is not limited to this. In the present first modification example, the guide mechanism 40 is provided on a surface different from the placement surface 32A of the decubitus table 30.

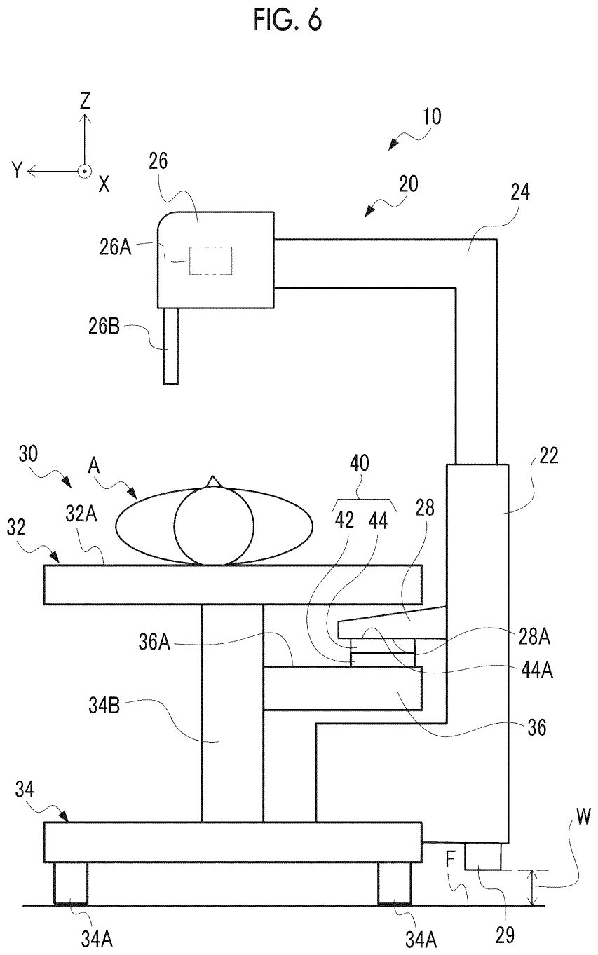

As shown in FIG. 6 as an example, the decubitus table 30 comprises a guide mechanism support portion 36. The guide mechanism support portion 36 is a member for supporting the guide mechanism 40. The guide mechanism support portion 36 is provided below the placement portion 32. The guide mechanism support portion 36 has a length of approximately the same length as the guide rail 42 of the guide mechanism 40.

In the example shown in FIG. 6, the guide mechanism support portion 36 is provided in an intermediate portion of the columnar portion 34B of the leg portion 34. The columnar portion 34B is attached to the lower surface of the placement portion 32 and is a columnar member extending in the up-down direction. The guide mechanism support portion 36 extends from an intermediate portion of the columnar portion 34B along the lateral direction of the placement portion 32. The guide mechanism support portion 36 is bridged between the columnar portions 34B of the pair of leg portions 34.

The guide mechanism 40 is provided on an upper surface 36A of the guide mechanism support portion 36. The attachment portion 28 of the radiation source unit 20 is provided at a position facing the stage member 44 of the guide mechanism 40 provided on the upper surface 36A of the guide mechanism support portion 36. In a case where the radiation source unit 20 is moved toward the decubitus table 30, the attachment portion 28 is attached to the stage member 44 of the guide mechanism 40 provided on the guide mechanism support portion 36. Here, the lower surface 28A of the attachment portion 28 is placed on the upper surface 44A of the stage member 44. The upper surface 36A is an example of a “surface different from the placement surface” according to the technique of the present disclosure.

Here, although a form example has described in which the guide mechanism support portion 36 is provided below the placement portion 32, this is merely an example. For example, the guide mechanism support portion 36 may be provided on a side surface of the placement portion 32. The guide mechanism support portion 36 has been described as an example provided in the columnar portion 34B, but this is merely an example. The guide mechanism support portion 36 may be attached to a dedicated member extending from a lower surface of the placement portion 32.

As described above, in the present first modification example, the guide mechanism 40 is provided on the upper surface 36A of the guide mechanism support portion 36 provided below the placement portion 32. Accordingly, the placement surface 32A is widened by the width of the guide mechanism 40. In addition, since the guide mechanism 40 has the guide rail 42 and the stage member 44, the guide mechanism 40 protrudes from the placement surface 32A by the amount of the members. In the present configuration, since the guide mechanism 40 is provided on the guide mechanism support portion 36, the placement surface 32A can be made flat.

### Second Modification Example

In the above-described embodiment, although a form example has been described in which the arm 24 extends from the upper surface of the main body portion 22 in the radiation source unit 20, the technology of the present disclosure is not limited to this. In the present second modification example, the arm 24 is provided on the side of the decubitus table 30 with respect to the main body portion 22. In this case, the attachment portion 28 is attachable to and detachable from the decubitus table 30 in a space formed by the upward movement of the arm 24.

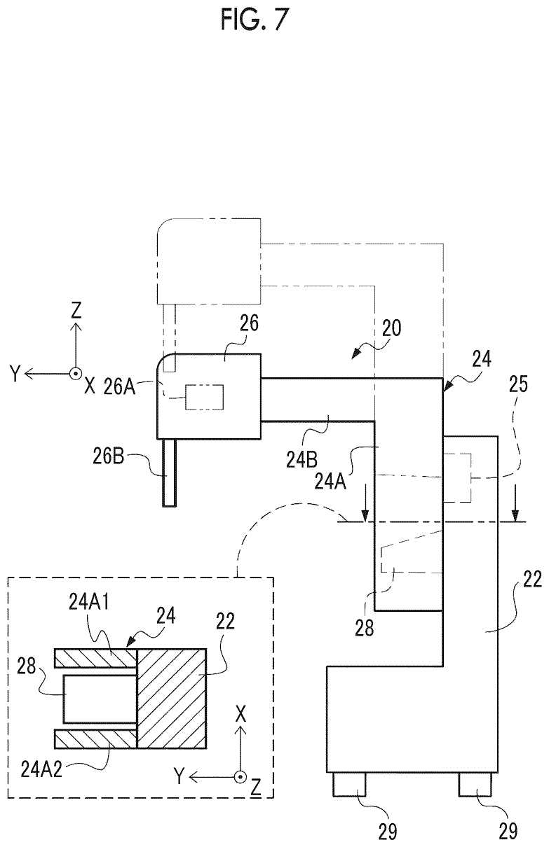

As shown in FIG. 7 as an example, the arm 24 comprises a vertical portion 24A along the up-down direction and a horizontal portion 24B extending from an upper end of the vertical portion 24A in the horizontal direction. The vertical portion 24A is attached to a surface of the main body portion 22 on the decubitus table 30 side (here, a left side surface). That is, the arm 24 is provided on the side of the decubitus table 30 closer to the main body portion 22. The main body portion 22 is an example of a “first site” according to the technique of the present disclosure, and the arm 24 is an example of a “second site” according to the technique of the present disclosure.

In addition, the arm 24 is movable in the up-down direction with respect to the main body portion 22 by the elevating mechanism 25. The elevating mechanism 25 is configured to include, for example, a power source such as a motor and a driving mechanism such as a feeding screw mechanism. In a state before the arm 24 rises with respect to the main body portion 22, a part of the attachment portion 28 is accommodated in the vertical portion 24A. Specifically, the vertical portion 24A includes a pair of wall portions 24A1 and 24A2. The pair of wall portions 24A1 and 24A2 are disposed to be spaced from each other in the front-rear direction (X direction shown in FIG. 7). The attachment portion 28 is accommodated between the pair of wall portions 24A1 and 24A2.

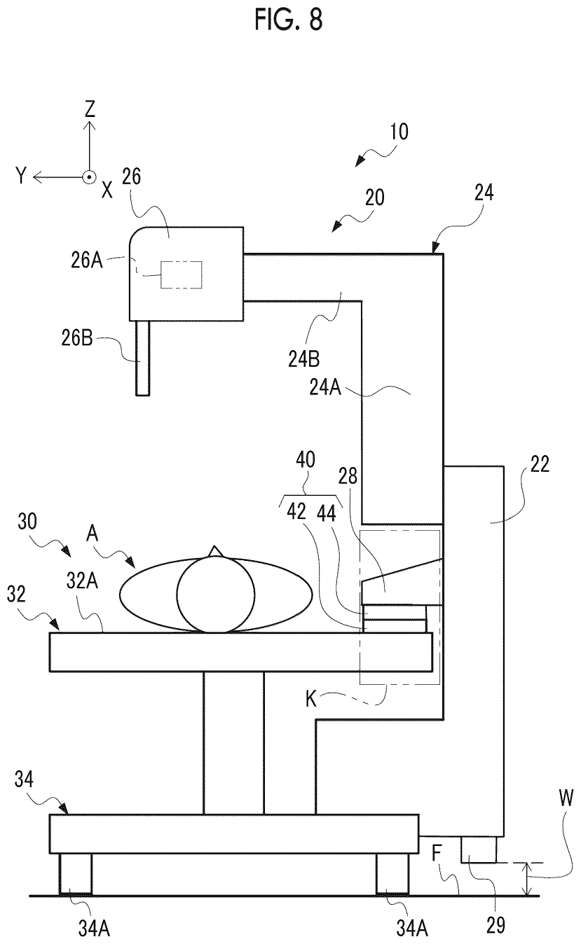

As shown in FIG. 8 as an example, in a case where the arm 24 is raised by the elevating mechanism 25, a space K is formed below the vertical portion 24A of the arm 24. In other words, the space K is a region in which the arm 24 is present before the arm 24 is raised in a case where the radiation source unit 20 is viewed from the front-rear direction. Then, the attachment portion 28 that is exposed by the arm 24 being raised is attached to the decubitus table 30. That is, in the space K, the attachment portion 28 is attachable to and detachable from the decubitus table 30.

Here, although a form example has been described in which the entire attachment portion 28 is accommodated in the vertical portion 24A of the arm 24, the technology of the present disclosure is not limited to this. For example, a part of the attachment portion 28 on the main body portion 22 side may be accommodated in the vertical portion 24A. In this case, all of the parts where the attachment and detachment between the attachment portion 28 and the decubitus table 30 are performed do not need to be included in the space K, and a part thereof may be included in the space K.

As described above, in the present second modification example, the arm 24 can be raised and lowered with respect to the main body portion 22, and the attachment portion 28 can be attached to and detached from the decubitus table 30 in the space K formed by the arm 24 being raised. Accordingly, the radiation source unit 20 can be attached to the decubitus table 30 in a state in which the radiation source unit 20 is close to the decubitus table 30 by the space of the vertical portion 24A of the arm 24. Accordingly, the centroid of the radiation source unit 20 can be brought close to the decubitus table 30, and the radiation source unit 20 can be easily moved.

In the present second modification example, although a form example has been described in which the arm 24 is directly attached to the main body portion 22, the technology of the present disclosure is not limited to this. For example, an intervention member may be provided between the arm 24 and the main body portion 22. The intervention member is allowed to be movable in the up-down direction with respect to the main body portion 22, and the arm 24 is allowed to be movable in the up-down direction with respect to the intervention member. In this case, the arm 24 and the intervention member move upward to form the space K. In addition, a so-called telescopic structure in which the arm 24 and the intervention member overlap each other in the contracted state may be employed.

In addition, in the present second modification example, the form example has been described in which the attachment portion 28 is accommodated between the pair of wall portions 24A1 and 24A2 of the vertical portion 24A, but the technology of the present disclosure is not limited to this. The attachment portion 28 may not interfere in the movement of the arm 24 in the up-down direction. For example, in a case where the arm 24 is positioned below, the attachment portion 28 may be accommodated inside the main body portion 22.

### Third Modification Example

In the above-described embodiment, the form example has been described in which the surface of the stage member 44 facing the attachment portion 28 is a flat surface, but the technology of the present disclosure is not limited to this. In the present third modification example, in the stage member 44, the roller member 46 is provided on a surface facing the attachment portion 28.

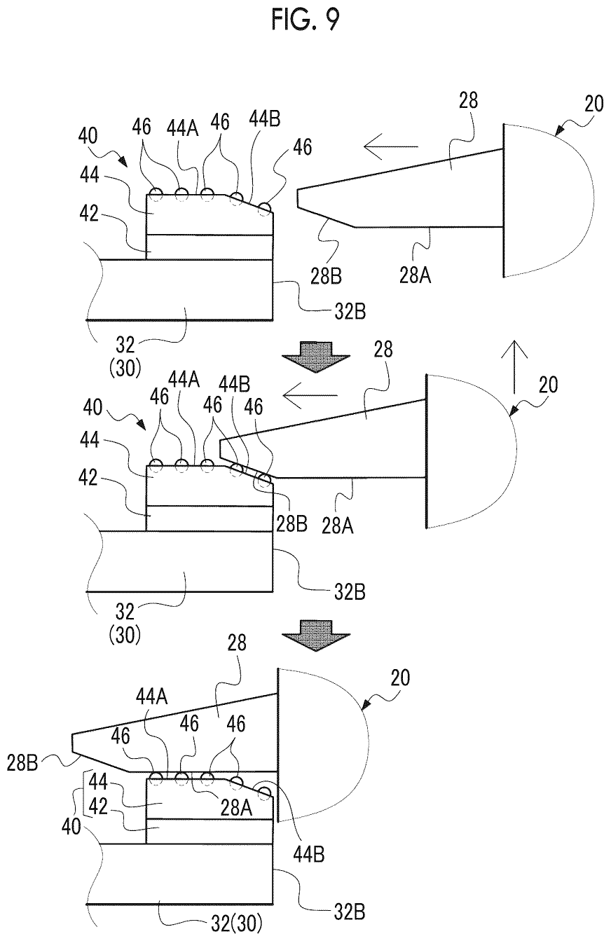

As shown in the upper portion of FIG. 9 as an example, the stage member 44 has a roller member 46 on a surface facing the attachment portion 28. Specifically, in a case where the attachment portion 28 is attached to the stage member 44, the roller member 46 is provided on a surface of the stage member 44 facing the attachment portion 28.

More specifically, the roller member 46 is provided on the upper surface 44A and the inclined surface 44B of the stage member 44. The roller member 46 is a cylindrical member having a rotation axis in a direction orthogonal to the attachment direction of the attachment portion 28. As shown in the middle part of FIG. 9, in a case where the attachment portion 28 comes into contact with the stage member 44, a roller member 46 provided on the inclined surface 44B comes into contact with the inclined surface 28B of the attachment portion 28 and is rotated. Further, as shown in the lower part of FIG. 9, in a case where the attachment portion 28 moves, a roller member 46 provided on the upper surface 44A comes into contact with the lower surface 28A of the attachment portion 28 and is rotated. As described above, the roller member 46 supports the movement of the attachment portion 28 in the attachment direction. The roller member 46 is an example of a “roller member” according to the technique of the present disclosure.

As described above, in the present third modification example, the roller member 46 is provided on the upper surface 44A and the inclined surface 44B of the stage member 44. Accordingly, in a case where the attachment portion 28 is attached, the roller member 46 is rotated in association with the contact with the attachment portion 28. Therefore, the attachment portion 28 can be easily attached to the stage member 44.

In the third modification example, although a form example has described in which the roller member 46 is provided on both the upper surface 44A and the inclined surface 44B, the technology of the present disclosure is not limited to this. For example, an aspect in which the roller member 46 is provided on any of the upper surface 44A and the inclined surface 44B may be adopted.

In addition, in the embodiment described above, the form example has been described in which the inclined surface 44B is formed on the stage member 44 and the inclined surface 28B is formed on the attachment portion 28, but the technology of the present disclosure is not limited to this. Both of the inclined surfaces 44B and 28B may not be formed, or any one of the inclined surfaces 44B and 28B may be formed.

In addition, in the embodiment described above, the form example has been described in which the attachment portion 28 is attached to the stage member 44 and the radiation source unit 20 rises with respect to the decubitus table 30 due to the inclined surface, but the technology of the present disclosure is not limited to this. For example, the radiation source unit 20 may be configured to include a mechanism that raises the radiation source unit 20 with respect to the decubitus table 30 after the attachment portion 28 is attached to the decubitus table 30.

In addition, in the embodiment described above, the form example has been described in which the guide rail 42 of the guide mechanism 40 is provided along the longitudinal direction of the decubitus table 30, but the technology of the present disclosure is not limited to this. For example, the guide rail 42 may be in an aspect of extending along the lateral direction of the decubitus table 30.

In addition, in the embodiment described above, the form example has been described in which the radiation source unit 20 is manually moved, but the technology of the present disclosure is not limited to this. For example, an aspect may be adopted in which the radiation source unit 20 is driven by a motor or the like.

In addition, in the embodiment described above, the form example has been described in which the decubitus table 30 is the dedicated bed for radiography, but the technology of the present disclosure is not limited to this. The decubitus table 30 may be a bed provided in a general ward or a general examination table. In this case, the guide mechanism 40 is separately attached to the decubitus table 30.

The described contents and the illustrated contents are detailed explanations of a part according to the technique of the present disclosure, and are merely examples of the technique of the present disclosure. For example, the descriptions regarding the configurations, the functions, the actions, and the effects are descriptions regarding an example of the configurations, the functions, the actions, and the effects of the part according to the present disclosed technology. Accordingly, in the contents described and the contents shown hereinabove, it is needless to say that removal of an unnecessary part, or addition or replacement of a new element may be employed within a range not departing from the gist of the present disclosed technology. In addition, in order to avoid complication and facilitate the understanding of a portion according to the present disclosed technology, regarding the contents described and illustrated above, description related to common technical knowledge or the like which does not need to be described to enable implementation of the present disclosed technology has been omitted.

In the present specification, “A and/or B” is synonymous with “at least one of A or B”. That is, “A and/or B” means that only A may be used, only B may be used, or a combination of A and B may be used. In the present specification, the same approach as “A and/or B” also applies to an expression of three or more matters connected with “and/or”.

All documents, patent applications, and technical standards disclosed in the present specification are incorporated in the present specification by reference to the same extent as those in a case where each of the documents, patent applications, and technical standards are specifically and individually indicated to be incorporated by reference.

Regarding the above-described embodiment, the following supplementary notes will be further disclosed.

### Supplementary Note 1

A radiography system including:
a decubitus table; and
a radiation source unit that is configured to be independently movable from the decubitus table and includes a radiation source,
in which the radiation source unit is capable of being attached to and detached from the decubitus table, and has an attachment portion that supports at least a part of a weight of the radiation source unit in a state in which the radiation source unit is attached to the decubitus table, and
the decubitus table has a guide mechanism that guides a movement of the radiation source unit attached through the attachment portion.

### Supplementary Note 2

The radiography system according to Supplementary Note 1,
in which, in a state in which the radiation source unit is attached to the decubitus table, the radiation source is positioned above the decubitus table, and a position of a centroid of the radiation source unit is positioned on the same side as the radiation source with respect to a side surface of the decubitus table facing the radiation source unit as viewed from a movement direction of the guide mechanism.

### Supplementary Note 3

The radiography system according to Supplementary Note 2,
in which the centroid of the radiation source unit is positioned on a radiation source side with respect to the attachment position.

### Supplementary Note 4

The radiography system according to any one of Supplementary Notes 1 to 3,
in which, in a state in which the radiation source unit is attached to the decubitus table, the radiation source unit is separated from a floor surface on which the decubitus table is installed.

### Supplementary Note 5

The radiography system according to any one of Supplementary Notes 1 to 4,
in which the attachment portion has a first inclined surface that is formed on a distal end side in an attachment direction of the radiation source unit and is inclined upward toward the distal end side.

### Supplementary Note 6

The radiography system according to any one of Supplementary Notes 1 to 5,
in which the guide mechanism includes
a guide rail that extends along the movement direction of the radiation source unit, and
a holding member that is movable along the guide rail, and
the attachment portion is attached to the holding member.

### Supplementary Note 7

The radiography system according to Supplementary Note 6,
in which the holding member is a flat plate-shaped stage member, and the attachment portion is attached to the decubitus table in a state in which a lower surface of the attachment portion is placed on an upper surface of the stage member.

### Supplementary Note 8

The radiography system according to Supplementary Note 6 or 7,
in which the holding member has a second inclined surface that is formed at an end portion on a radiation source unit side and is inclined downward toward the radiation source unit side.

### Supplementary Note 9

The radiography system according to any one of Supplementary Notes 6 to 8,
in which the holding member includes a roller member on a surface facing the attachment portion.

### Supplementary Note 10

The radiography system according to any one of Supplementary Notes 1 to 9,
in which the radiation source unit has a first site and a second site that is provided on a side of the decubitus table with respect to the first site and is movable in an up-down direction with respect to the first site, and
the attachment portion is attachable to and detachable from the decubitus table in a space formed by upward movement of the second site.

### Supplementary Note 11

The radiography system according to any one of Supplementary Notes 1 to 10,
in which a placement surface, which is a surface on which the subject is placed in the decubitus table has a rectangular parallelepiped shape in a plan view, and
a movement direction of the guide mechanism is along a longitudinal direction of the placement surface.

### Supplementary Note 12

The radiography system according to any one of Supplementary Notes 1 to 11,
in which the guide mechanism is provided on a surface different from a placement surface, which is a surface on which the subject is placed in the decubitus table.

What is claimed is:

1. A radiography system comprising:
a decubitus table; and
a radiation source unit that is configured to be independently movable from the decubitus table and includes a radiation source,
wherein the radiation source unit is capable of being attached to and detached from the decubitus table, and has an attachment portion that supports at least a part of a weight of the radiation source unit in a state in which the radiation source unit is attached to the decubitus table, and
the decubitus table has a guide mechanism that guides a movement of the radiation source unit attached through the attachment portion.

2. The radiography system according to claim 1,
wherein, in a state in which the radiation source unit is attached to the decubitus table, the radiation source is positioned above the decubitus table, and a position of a centroid of the radiation source unit is positioned on the same side as the radiation source with respect to a side surface of the decubitus table facing the radiation source unit as viewed from a movement direction of the guide mechanism.

3. The radiography system according to claim 2,
wherein the centroid of the radiation source unit is positioned on a radiation source side with respect to the attachment position.

4. The radiography system according to claim 1,
wherein, in a state in which the radiation source unit is attached to the decubitus table, the radiation source unit is separated from a floor surface on which the decubitus table is installed.

5. The radiography system according to claim 1,
wherein the attachment portion has a first inclined surface that is formed on a distal end side in an attachment direction of the radiation source unit and is inclined upward toward the distal end side.

6. The radiography system according to claim 1,
wherein the guide mechanism includes
a guide rail that extends along the movement direction of the radiation source unit, and
a holding member that is movable along the guide rail, and
the attachment portion is attached to the holding member.

7. The radiography system according to claim 6,
wherein the holding member is a flat plate-shaped stage member, and
the attachment portion is attached to the decubitus table in a state in which a lower surface of the attachment portion is placed on an upper surface of the stage member.

8. The radiography system according to claim 6,
wherein the holding member has a second inclined surface that is formed at an end portion on a radiation source unit side and is inclined downward toward the radiation source unit side.

9. The radiography system according to claim 6,
wherein the holding member includes a roller member on a surface facing the attachment portion.

10. The radiography system according to claim 1,
wherein the radiation source unit has a first site and a second site that is provided on a side of the decubitus table with respect to the first site and is movable in an up-down direction with respect to the first site, and
the attachment portion is attachable to and detachable from the decubitus table in a space formed by upward movement of the second site.

11. The radiography system according to claim 1,
wherein a placement surface, which is a surface on which the subject is placed in the decubitus table has a rectangular parallelepiped shape in a plan view, and
a movement direction of the guide mechanism is along a longitudinal direction of the placement surface.

12. The radiography system according to claim 1,
wherein the guide mechanism is provided on a surface different from a placement surface, which is a surface on which the subject is placed in the decubitus table.
